(19) United States
(12) Patent Application Publication
(10) Pub. No.: US 2016/0157793 A1
(43) Pub. Date: Jun. 9, 2016

(54) DEVICE TO ASSIST WITH AN EXAMINATION OF THE KNEE

(71) Applicants: Michel DESROSES, Torcy (FR); Patrick BENSAID, Bailly-Romainvilliers (FR); El hadj SABARA, Lognes (FR)

(72) Inventors: Michel DESROSES, Torcy (FR); Patrick BENSAID, Bailly-Romainvilliers (FR); El hadj SABARA, Lognes (FR)

(21) Appl. No.: 14/906,344

(22) PCT Filed: Jul. 22, 2014
(86) PCT No.: PCT/FR2014/051891

§ 371 Date: Jan. 20, 2016

(30) Foreign Application Priority Data
Jul. 22, 2013 (FR) 1357179

(51) Int. Cl. A61B 6/04

(52) U.S. Cl. CPC A61B 6/0421; A61B 6/0457

(57) ABSTRACT

A device (1) for placing the joint of the knee of a person in contraction with a view to performing an examination of the said knee. The device comprises: a structure (2) adapted to receive the person in a seated or recumbent position, locking means (4) for locking a corresponding thigh of the person with respect to the structure, and a toe-clip (5_{D}, 5_{G}), adapted to receive a corresponding foot of the person, the said toe-clip comprising means for locking the foot of the person and being rotatable about an axis (R_{D}, R_{G}) adapted to be placed perpendicular to the foot of the person.

The present invention concerns a device for placing the joint of the knee of a person in contraction with a view to performing an examination of the said knee.

Radiography is a transmission imaging technique, using X-rays in the context of X-radiography. Radiography has the advantage of being rapid and low in cost.

However, radiography permits only a limited number of types of diagnosis. In particular, in the case of the knee, radiography can make it possible to diagnose a fracture, but not other types of injuries, such as a patellar lesion (for example, a luxation).

Consequently, it is often necessary to supplement radiographic examination of the knee with other techniques, particularly magnetic resonance imaging (MRI).

However, the conduct of an MRI scan is a costly procedure, generally involving long waiting times. Moreover, a single MRI scan does not always allow a reliable diagnosis to be made, and this may lead to unnecessary surgical operations and/or the conduct of additional MRI scans after injecting a special substance into the patient.

There is therefore a need for devices that allow the knee to be examined in a rapid and economic manner, while making it possible to obtain a reliable diagnosis. The present invention aims to improve the situation.

To this end, the invention proposes a device for placing the joint of a knee of a person in contraction, with a view to performing an examination of the said knee. The device comprises:
a structure intended to receive the person in a seated or recumbent position,
locking means for locking a corresponding thigh of the person with respect to the structure,
a toe-clip intended to receive a corresponding foot of the person, the said toe-clip comprising means for locking the foot of the person and being rotatable about an axis intended to be placed perpendicular to the foot of the person, the said toe-clip being capable of being locked in a first position, in which the foot of the person is locked in a position of outward rotation, and in a second position, in which the foot of the person is locked in a position of inward rotation.

The structure may comprise a seat. In this case, the device may further comprise means for adjusting a distance between the seat and the toe-clip.

The device may be made from one or more materials suitable for the performance of a radiography examination. The said material(s) may comprise, for example, carbon fibre.

The device may also comprise a cassette-holder.

The plane of the toe-clip may be rotatable about an axis intended to be placed perpendicular to the corresponding tibia of the person.

The device may comprise:
means for locking the two thighs of the person with respect to the structure,
two toe-clips, intended to receive respectively the two feet of the person.

The device may also comprise drive means.

Other features and advantages of the invention will become clear from a reading of the description that follows, which is purely illustrative in nature and must be read in the light of the annexed drawings, wherein:

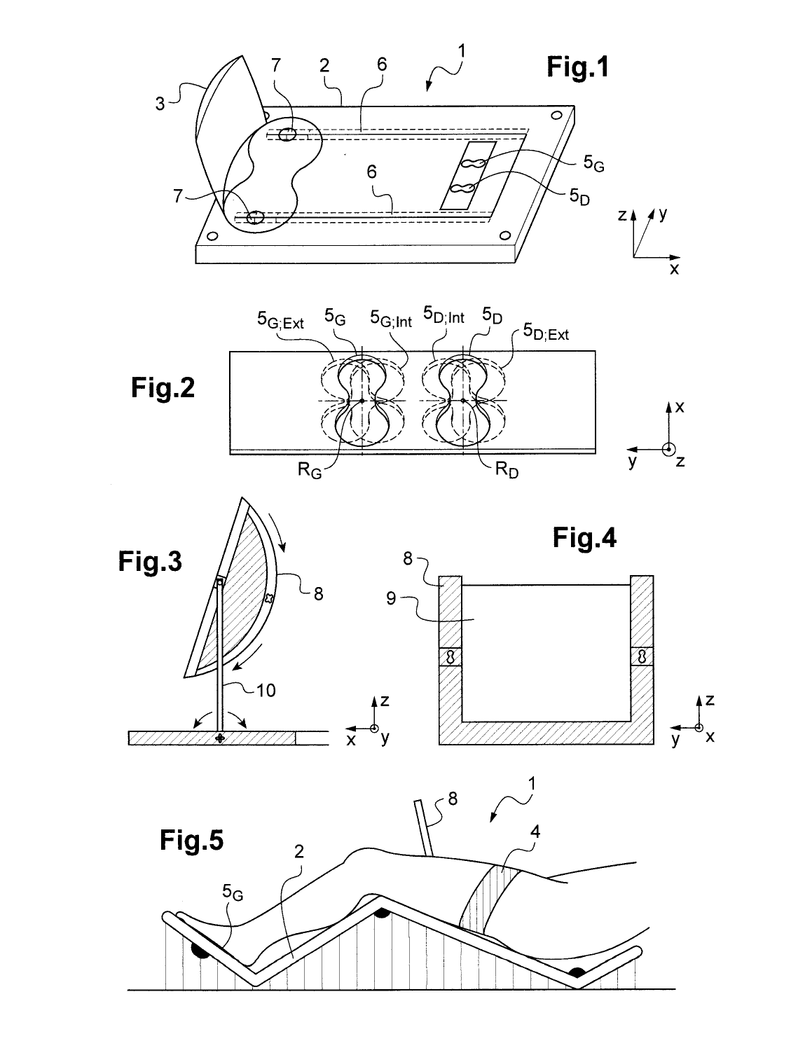

FIG. 1 is a schematic perspective view of a device for placing the joint of the knee in contraction according to a first embodiment of the invention;

FIG. 2 is a top view of the toe-clip of the device of FIG. 1;

FIG. 3 is a profile view of a cassette-holder;

FIG. 4 is a front view of the cassette-holder of FIG. 3; and

FIG. 5 is a profile view of a device for placing the knee in contraction according to a second embodiment of the invention.

FIG. 1 represents a first embodiment of device 1 for placing the joint of the knee of a person in contraction.

The purpose of the device 1 is to facilitate an examination of the knee. The examination is intended to be performed with the aid of an imaging technique, for example radiography, magnetic resonance imaging (MRI), tomography or ultrasound scanning.

The device 1 is therefore made from one or more materials suitable for the implementation of imaging techniques, particularly radiography. The material(s) may comprise, for example, carbon fibre.

The device 1 comprises a structure 2, intended to receive the person in a seated, semi-recumbent or recumbent position. According to the embodiment represented in FIG. 1, the person is placed in a seated position. According to a second embodiment represented in FIG. 5, the person is placed in a semi-recumbent position.

The structure 2 may further comprise a seat 3, to improve the comfort of the person.

The device 1 further comprises locking means 4 for locking a thigh of the person with respect to the structure 2.

The locking means are visible in FIG. 5. The locking means 4 comprise, for example, a self-gripping strip of the Velcro® type.

Preferably, the device 1 comprises means for locking the two thighs of the person with respect to the structure 2. Thus, the device 1 is suitable for an examination of the right knee and/or the left knee.

The device 1 further comprises at least one toe-clip 5, intended to receive a foot of the person.

As indicated above, the device 1 preferably comprises two toe-clips 5_{D} and 5_{G} (more clearly visible in FIG. 2), to allow an examination of the right knee and/or the left knee.

The device 1 may further comprise adjusting means 6 for adjusting a distance between the seat 3 and the toe-clips 5_{D} and 5_{G}. The adjustment distance depends, among other things, on a desired flexing of the knee of the person. The desired flexing may be, for example, 30°, 45°, 60° or 90°. The adjusting means 6 may be adjusted during the course of the examination, according to the height of the person and the desired flexing.

According to the embodiment represented in FIG. 1, the adjusting means comprise two guides 6 arranged parallel to each other between the seat 3 and the toe-clips 5_{D} and 5_{G}. The seat 3 is suitable for sliding along the two guides 6, and for being fixed at a chosen distance from the toe-clips 5_{D} and 5_{G} with the aid of screws 7.

Other adjusting means may be provided. For example, two rods, each comprising notches, may be arranged parallel to each other between the seat 3 and the toe-clips 5_{D} and 5_{G}. The seat 3 is then adjusted to engage with a pair of notches opposite to each other, and the choice of the pair of notches allows adjustment of the distance.

Each toe-clip 5_{D}, 5_{G} comprises locking means (not shown) for locking the foot. The locking means comprise, for example, self-gripping strips.

Each toe-clip 5_{D}, 5_{G} is rotatable about an axis R_{D}, R_{G} intended to be placed perpendicular to the foot of the person. The axes R_{D} and R_{G} are parallel to the axis Z in FIG. 2.

Each toe-clip 5_{D}, 5_{G} is suitable for being locked in a first position, in which the foot of the person is locked in a position of outward rotation. The positions of outward rotation 5_{D,Ext}, 5_{G,Ext} are represented by dotted lines in FIG. 2.

Each toe-clip 5_{D}, 5_{G} is also suitable for being locked in a second position, in which the foot of the person is locked in a position of inward rotation. The positions of locking in inward rotation 5_{D,Int}, 5_{G,Int} are represented by dotted lines in FIG. 2. The locking is effected, for example, by using clipping means.

The plane of the toe-clips 5_{D}, 5_{G} may be rotatable about an axis intended to be placed perpendicular to the tibias of the person. The axis of rotation is parallel to the axis Y in FIG. 1. This may make it possible to improve the comfort of the person, particularly when the person has osteoarthritis. The angle of inclination may be, for example, approximately 15°.

As represented in FIGS. 3 and 4, the device 1 may further comprise a holder 8 for a cassette 9. In a manner that is known per se, in order to perform a radiography, a cassette 9 comprising a screen/film pair is placed behind the object to be radiographed. When a person is placed in a recumbent or semi-recumbent position, it may be difficult for him or her to hold the cassette 9 in the proper position. The holder 8 may thus be fixed to the ground or to the structure 2, for example with the aid of rods 10. The cassette 9 can then be held in place without any intervention from the person.

The device 1 may further comprise drive means and/or automation means (not shown). The automation means are preferably wireless, so as not to interfere with the magnetic field during an MRI.

The device 1 may further comprise supporting means (not shown), allowing it to be supported on a knee and/or an ankle of the person, for example in the case of valgus or varus.

There follows a description of the operation of the device 1 for performing an examination of the right knee of a person.

The person is placed on the seat 3. If necessary, the position of the seat 3 is adjusted. The person then places his or her right foot in the toe-clip 5_{D}.

The right thigh of the person is then fixed with respect to the structure 2, and his or her right foot is fixed in the toe-clip 5_{D}.

If the examination is a radiography, the holder 8 and the cassette 9 are put in place.

The practitioner then asks the person to turn his or her right foot inwards or outwards. Since the foot of the person is fixed, the joint of the right knee works. The toe-clip 5_{D} is locked in the desired position for the time required to perform the examination.

The contraction of the muscles of the lower limb makes it possible to assess the muscular, ligamentous and capsular system of the examined knee. The device 1 thus allows a simple and reliable analysis of pains of the chronic inflammation type. It also allows the diagnosis of patellar lesions, for example a patellar luxation or subluxation, and/or muscular, capsular or tendinous tears.

With the device 1, a radiography is generally sufficient to obtain a reliable diagnosis. It is thus possible to reduce costs and waiting times.

Of course, the present invention is not limited to the embodiments described above by way of example, but also extends to other variants.

1. A device for placing the joint of the knee of a person in contraction with a view to performing an examination of the said knee, wherein the device comprises:
a structure adapted to receive the person in a seated or recumbent position;
locking means for locking a corresponding thigh of the person with respect to the structure; and
a toe-clip configured to receive a corresponding foot of the person, said toe-clip comprising means for locking the foot of the person and being rotatable about an axis adapted to be placed perpendicular to the foot of the person, said toe-clip being lockable in a first position, in which the foot of the person is locked in a position of outward rotation, and in a second position, in which the foot of the person is locked in a position of inward rotation.

2. The device according to claim 1, wherein the structure comprises a seat.

3. The device according to claim 2, further comprising adjusting means for adjusting a distance between the seat and the toe-clip.

4. The device according to claim 1, made from one or more materials suitable for the performance of a radiography examination, said material(s) comprising carbon fibre.

5. The device according to claim 4, further comprising a holder for a cassette.

6. The device according to claim 1, wherein the plane of the toe-clip is rotatable about an axis adapted to be placed perpendicular to the corresponding tibia of the person.

7. The device according to claim 1, comprising:
locking means for locking the two thighs of the person with respect to the structure; and
two toe-clips adapted to receive respectively the two feet of the person.

8. The device according to claim 1, further comprising drive means.
